(19) United States
(12) Patent Application Publication
(10) Pub. No.: US 2018/0177457 A1
(43) Pub. Date: Jun. 28, 2018

(54) Nerve Monitoring Endotracheal Tube

(71) Applicant: Xialing Zhang, Mountain View, CA (US)

(72) Inventor: Xialing Zhang, Mountain View, CA (US)

(21) Appl. No.: 15/803,812

(22) Filed: Nov. 5, 2017

Related U.S. Application Data
(60) Provisional application No. 62/439,888, filed on Dec. 28, 2016.

(51) Int. Cl. A61B 5/00; A61B 5/0492; A61B 5/04; A61M 16/04

(52) U.S. Cl. CPC A61B 5/6852; A61B 5/0492; A61M 2205/3303; A61M 16/0402; A61B 5/04001

(57) ABSTRACT

An endotracheal tube electrode assembly comprises an elongated electrode body, a plurality of electrodes and a layer of insulating material. The elongated electrode body has a semi-circular cross-sectional shape defining an endotracheal tube-receiving channel that extends along a length of the elongated electrode body. The plurality of electrodes is provided on a surface of the elongated electrode body outside of the endotracheal tube-receiving channel. The electrodes extend along a length of the elongated electrode body. Each of the electrodes is formed from a conductive material applied directly onto the elongated electrode body. The layer of insulating material formed on the elongated electrode body covering a portion of each one of the electrodes, thereby providing an uncovered portion of the at least one pair of electrodes to serve as a patient contacting thereof.

### CROSS-REFERENCE TO RELATED APPLICATIONS

This non-provisional patent application claims priority from co-pending U.S. Provisional Patent Application having Ser. No. 62/439,888, filed 28 Dec. 2016, entitled “NERVE MONITORING ENDOTRACHEAL TUBE”, having a common applicant herewith and being incorporated herein in its entirety by reference.

### FIELD OF THE DISCLOSURE

The disclosures made herein relate generally to medical devices and, more particularly, to endotracheal tubes adapted to enable monitoring of nerve signals.

### BACKGROUND

Many surgical procedures around the neck such as thyroid surgeries, cervical spine surgeries have potentials of damage on the recurrent laryngeal nerve. Similarly, craniotomies can cause damage to the vagus nerve, which is the 10th cranial nerve. The vagus nerve is parasympathetic. Innervates the smooth muscle of the trachea, bronchi. Surgical damage of recurrent laryngeal nerve and/or vagus nerve generally lead to a patient experiencing difficulty in swallowing and hoarseness of voice.

Placement of an endotracheal tube is a good approaching of recording bio-signals from the muscle groups of trachea and vocal cords. More specifically, a nerve monitoring endotracheal tube is placed into patient's airway in accordance with well-known endotracheal tube placement procedures and is used in combination with a neuromonitoring equipment for monitoring neural functional activities (e.g., nerve signals) during a surgical procedure. Such monitoring serves the purpose of attempting to prevent neural functional injury caused by possible surgical manipulation and enhancing surgical safety index by lowering surgical risk of neuron injury caused by surgical damage.

Prior art nerve monitoring endotracheal tubes include electrodes that are formed (e.g., painted) directly on an exterior surface of the endotracheal tube. For example, a prior art nerve monitoring endotracheal tubes disclosed in U.S. Pat. No. 8,634,894 has a silver-containing material composition painted directly on a conventional endotracheal tube, which during such painting is subjected to relatively high temperature and can thus have potential adverse side effects on materials of endotracheal tube. Furthermore, such prior art endotracheal tubes are made from a relatively hard and stiff material to provide for sufficient rigidity for allowing the endotracheal tube to retain its cross-sectional shape and longitudinal contour when placed within a patient's airway. Due to such harness and stiffness, as well as the materials from which endotracheal tubes are made, it is difficult to form the conductive material of the electrodes on the endotracheal tube in a manner that does not exhibit cracking from even minimal flexing and bending. Such cracks cause poor quality of conductive signals being transferred through from the nerves to the neuromonitoring equipment.

Therefore, a nerve monitoring endotracheal tube that overcomes drawbacks associated with conventional nerve monitoring endotracheal tubes would be advantageous, desirable and useful.

### SUMMARY OF THE DISCLOSURE

Embodiments of the present invention are directed to nerve monitoring endotracheal tubes. More specifically, embodiments of the present invention are directed to a novel construction of nerve monitoring endotracheal tubes that employs an electrode configuration providing resistance to cracking of such electrodes. This resistance to cracking of the electrodes is beneficial because it provides for high quality of conductive signals being transferred from the nerves through such an endotracheal tube to neuromonitoring equipment acquiring such signals.

In one embodiment of the present invention, an endotracheal tube comprises a tubular body, an electrode body and a plurality of electrodes. The tubular body has a proximate end portion and a distal end portion. A central passage extends contiguously through the proximate and distal end portions of the tubular body. The electrode body is attached at a plurality of locations thereof to an outside surface of the tubular body. The electrode body extends length-wise from the distal end portion toward the proximate end portion. The plurality of electrodes is provided on and extending along a length of the electrode body.

In another embodiment of the present invention, an endotracheal tube comprises a tubular body and one or more electrode assemblies. The tubular body has a proximate end portion and a distal end portion. A central passage extends contiguously through the proximate and distal end portions of the tubular body. The one or more electrode assemblies each includes an elongated electrode body having opposing major surfaces. Each of the one or more electrode assemblies has at least one pair of electrodes extending lengthwise on a first one of the major surfaces of the elongated body thereof. The elongated body of each of the one or more electrode assemblies is engaged at plurality of locations along a length of the tubular body. A second one of the major surfaces of the elongated body of each of the one or more electrode assemblies faces an exterior surface of the tubular bod. Each one of the electrode assemblies includes a layer of insulating material formed on the elongated electrode body thereof. The layer of insulating material covers a portion of each electrode of each one of said pairs of electrodes, thereby providing an uncovered portion of the at least one pair of electrodes to serve as a patient contacting thereof.

In another embodiment of the present invention, an endotracheal tube electrode assembly comprises an elongated electrode body, a plurality of electrodes and a layer of insulating material. The elongated electrode body has a semi-circular cross-sectional shape defining an endotracheal tube-receiving channel that extends along a length of the elongated electrode body. The plurality of electrodes is provided on a surface of the elongated electrode body outside of the endotracheal tube-receiving channel. The electrodes extend along a length of the elongated electrode body. Each of the electrodes is formed from a conductive material applied directly onto the elongated electrode body. The layer of insulating material formed on the elongated electrode body covering the electrodes. A portion of each electrode adjacent to each other one of the electrodes is not covered by the layer of insulating material thereby forming a patient contacting portion of the electrodes.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a perspective view showing an endotracheal tube configured in accordance with an embodiment of the present invention.

FIG. 2 is a cross-sectional view taken along the line 2-2 in FIG. 1.

FIG. 3 is a cross-sectional view taken along the line 3-3 in FIG. 1.

FIG. 4 is a cross-sectional view taken along the line 4-4 in FIG. 1.

### DETAILED DESCRIPTION

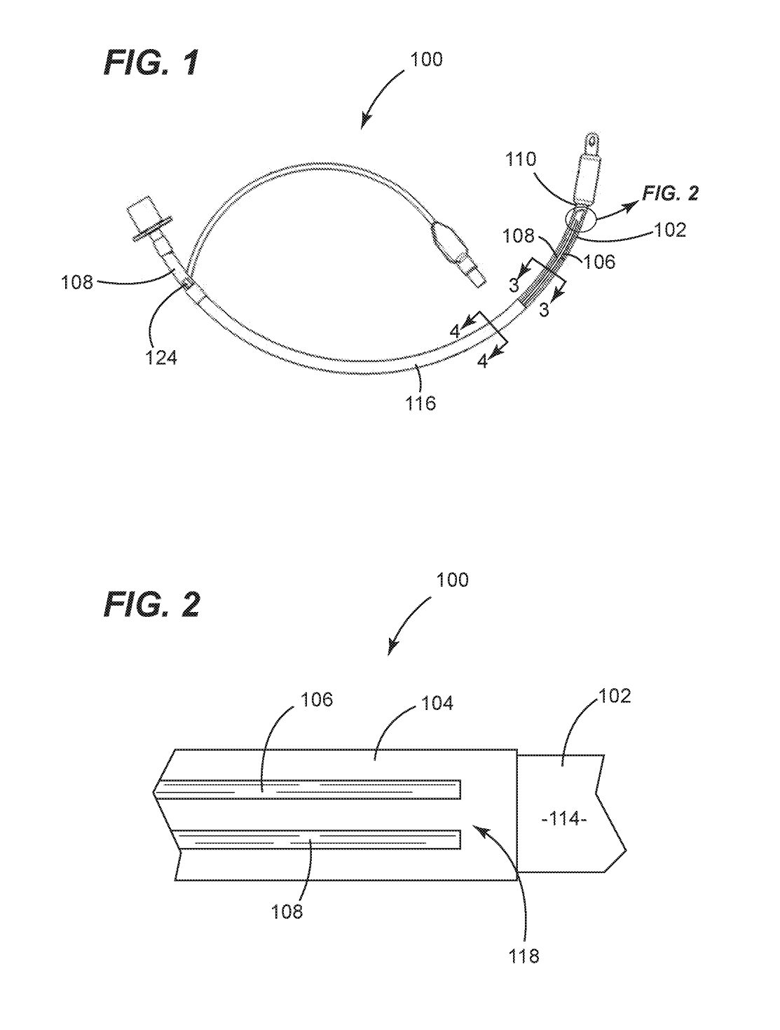

FIG. 1 shows an endotracheal tube 100 configured in accordance with an embodiment of the present invention. As discussed herein in greater detail, the endotracheal tube 100 enables monitoring of signal generated by a human body (i.e., a host organism) within which the endotracheal tube 100 is placed. Such monitoring is enabled by a novel construction of electrodes that are integral with the endotracheal tube 100. The novel construction includes an electrode configuration that provides resistance to cracking of such electrodes, which is beneficial because it provides for high quality of conductive signals being transferred from the nerves through the endotracheal tube 100 to neuromonitoring equipment acquiring such signals. To this end, important nerve functions can be constantly monitored during the surgeries, giving patients and surgeons a safer surgical guidance.

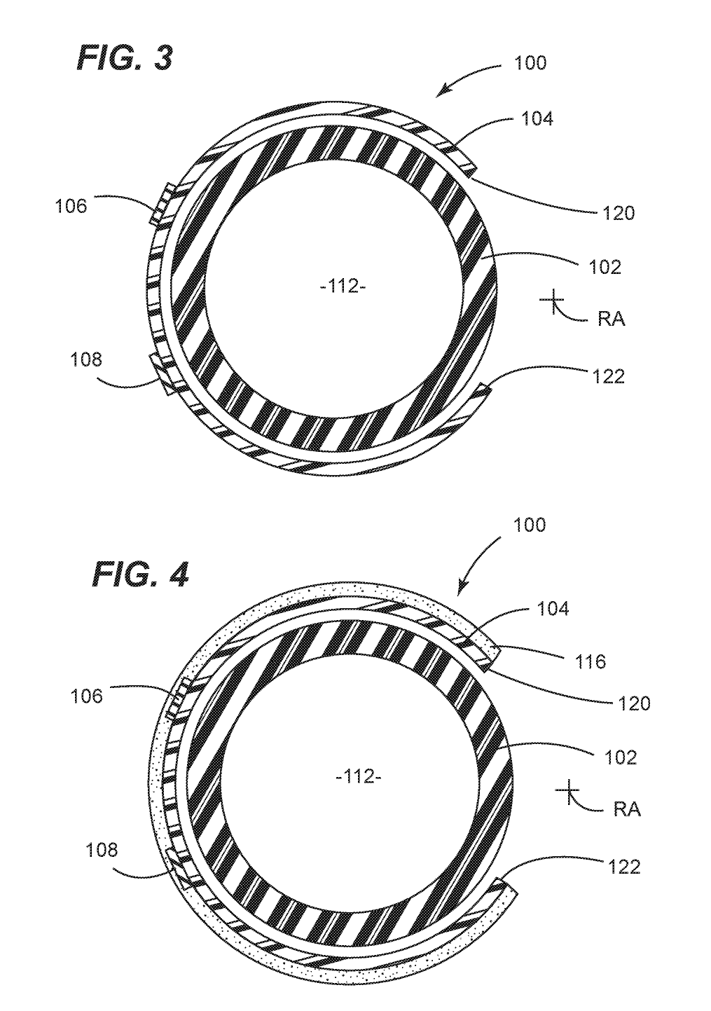

Referring now to FIGS. 1-3, the endotracheal tube 100 comprises a tubular body 102, an electrode body 104 and a plurality of electrodes 106, 108. The tubular body 102 has a proximate end portion 109 and a distal end portion 110. A central passage 112 (as shown in FIGS. 3 and 4) extends contiguously through the proximate and distal end portions 109, 110 of the tubular body 102. In some embodiments, the tubular body 102 is that of a conventional (e.g., commercially available) endotracheal tube that is not otherwise configured for enabling monitoring of electrical signals generated by a human body (e.g., recurrent laryngeal nerve signals and/or vagus nerve signals).

The electrode body 104 is attached at a plurality of locations thereof to an outside surface 114 (FIG. 2) of the tubular body 102. The electrode body 104 extends length-wise from the distal end portion 110 toward the proximate end portion 109. The plurality of electrodes 106, 108 is provided on and extending along a length of the electrode body 104, thereby providing an electrode assembly. In preferred embodiments, the electrode body 104 is made from a thin and flexible layer of material. Preferably, such electrode body material is a polymeric material offering substantial non-conductivity. Various commercially-available polymeric films and sheets are suitable for use as the electrode body 104.

A layer of insulating material 116 is formed on the electrode body 104. The layer of insulating material 116 covers a portion of each of the electrodes 106, 108. A portion of the electrodes 106, 108 are no covered by the layer of insulating material 116, thereby forming a patient contacting portion 118 that is able to contact patient target muscle groups directly for recording bio-signals therefrom. It is disclosed herein that the layer of insulating material 116 not covering the electrodes 106, 108 can be an area outside of a region in which the layer of insulating material 116 is provided (e.g., a region of the electrode body 104 that is not coated with the layer of insulating material 116) or can be a region that is devoid of the layer of the insulating material 116 but is otherwise surrounded by the layer of the insulating material 116.

The electrodes 106, 108 are formed on the electrode body 104. In a preferred embodiment, the electrodes 106, 108 are formed onto the electrode body 104 from a respective conductive material. In one specific embodiment, the electrodes 106, 108 are formed from a conductive material applied directly onto the electrode body 104. Examples of such conductive materials include, but are not limited to, Graphene, silver and other similarly biostable, conductive materials offering desirable electrical conductivity. Laser spray is a commercially-available material deposition technique suitable for applying Graphene, silver and the like onto a base material such as the electrode body 104. Such laser spray deposition technique offers superior deposition control than traditional painting of such materials. It is disclosed herein that electrodes in accordance with embodiments of the present invention can be formed using a variety of known and yet to be discovered materials and/or techniques for creating electrically conducting structures on polymeric substrates.

Advantageously, electrodes and the electrode body of electrode assemblies configured in accordance with embodiments of the present invention are jointly made from highly compatible materials. Such compatibility refers to the material of the electrodes and the electrode body providing resistance to defect such as delamination cracks and the like, thereby providing better longevity in and quality of signal being generated via the electrodes. Thermoplastic polyurethane (TPU) and materials having similar thermal expansion properties and physical properties are preferred materials for the electrode body.

The material compatibility is beneficial because it resolves a well-known existing problem of prior art electrode-carrying endotracheal tubes in which conductive electrode material develops cracks due to poor material compatibility between the prior art endotracheal tube material and the conductive printing ink from which prior art electrodes are formed. The defects that develop between such prior art electrodes and endotracheal tube material adversely impact the signal quality. However, stable recording signals are critical for surgical procedures utilizing electrode-carrying endotracheal tubes such as those configured in accordance with embodiment of the present invention.

The electrodes 106, 108 are formed on the electrode body 104 as two conductive structures that are separated by space for providing electrical isolation therebetween. For example, relative to a reference axis that is mid-way between terminal edges 120, 122 of the electrode body 104, the electrodes 106, 1080 are each about 150 degrees (in opposite directions) from such reference axis RA. Accounting for width of the electrodes 106. 108, the can be about a 30-degree space therebetween. In other embodiments, there will be different spacing between the electrodes, with of the electrodes and/or angular positioning of the electrodes.

Each of electrodes 106, 108 defines a respective electrical channel—e.g., the electrode 106 forming a positive (plus (+)) electrical channel and the electrode 108 forming a negative (minus (−)) electrical channel. Preferably, a plurality of pairs of electrodes are provided on the electrode body. Each pair of electrode is partially covered by a layer of the insulating material to define a respective patient contacting portion for contacting a patient to produce a bio-signal. Electrodes 106, 108 terminate into a printed circuit board (PCB 124), which serves as a connector between the electrodes and wire leads (not shown) having a standard plug compatible with a neuromonitoring system. In some embodiments, carbon fiber wire can be used as wire lead to connect with electrodes 106, 108 to the PCB 124. The PCB 124 provides the convenience of allowing the electrode assembly of the endotracheal tube 100 to be connected to the neuromonitoring system (i.e., a recording unit) when pre-operation patient setup is complete and to be disconnected therefrom when the surgery is ended. In this respect, the PCB 120 serves to avoids disruption of a clinician's work prior to, during and after patient setup.

The electrodes 106, 108 and the electrode body 104 jointly provide an electrode assembly that serves as a signal acquiring membrane that covers a portion of tubular body of an endotracheal tube. As shown in FIGS. 2 and 3, the electrode body 104 of such electrode assembly has a semi-circular cross-sectional shape. The electrode body is wrapped around the tubular body 102. In some embodiments, the tubular body 104 is that of a commercially-available endotracheal tube whereby a ready-to-use nerve monitoring endotracheal tubes is provided or an electrode assembly in accordance with embodiments of the present invention can be manually attached to an off-the-shelf endotracheal tube such as by a medical professional who will subsequently place the endotracheal tube in a patient.

Advantageously, an electrode assembly configured in accordance with an embodiment of the present invention reduces the potential for cracking of the electrodes thereof. One reason for such reduced potential for cracking of the electrodes is that the can be made from a material that offers superior properties as a structural base for electrodes. The electrode body 104 can be made from a thinner, more flexible material than the tubular body 102 that serves as the airway for the endotracheal tube 100. The material from which the electrode body 104 is made can also be selected from a material that offers superior electrode material adhesion that the material from which the tubular body 102 is made. Another reason for such reduced potential for cracking of the electrodes is that the electrode body 104 serves as a “membrane sheet” that is engaged with tubular body 102, but that is decoupled from bending-induced stresses from deformation of/applied loads of the tubular member. The electrode body 104 is preferably engaged with the tubular member at only a few discrete locations. For example, the electrode body 10 can be secured to the tubular body 102 (e.g., by thermal bonding, adhesive bonding, mechanical attachment, and/or the like) only at opposing ends of the electrode body 104, at one or more discrete locations between such opposing ends or a combination thereof). In this respect, the electrode body 104 is preferably isolated from loads and deformation of the tubular member whereby bending-induced stresses from deformation of/applied loads of the tubular member are not directly/fully transmitted in to the electrode body 104. However, in some embodiments, the electrode body 104 can be secured to the tubular member over substantially an entire area of the electrode body 104. (e.g., major surface of the electrode body being bonded/adhered to the tubular body 102).

In use, the endotracheal tube 100 is placed within the endotracheal airway of a patient. The electrodes of the 106, 108 come into contact with the target tissue (e.g., lal 12ryngeal nerves and/of vagus nerves), whereby the electrode assembly acts as a signal acquisition device to acquire their bio-signals (e.g., muscle electromyograph (EMG) activities) and enable such bio-signals to be analyzed in a neuromonitoring system to verify nerve functional involvement so as to avoid damage to the target tissue during a surgical procedure. In this respect, endotracheal tubes and electrode assemblies configured in accordance with embodiment of the present invention can serve as recording devices that acquire signals from direct contact with the trachea and vocal cords. For example, in preferred embodiments, the electrode body of an electrode assembly wraps around the outside of the tubular body of an endotracheal tube and electrodes on the electrode body act as recording channels that directly contact a patient's vocal cord and tracheal muscle groups thereby acquiring EMG activities that reflect innervating neural functions of, for example, vagus nerves and recurrent laryngeal nerves. Monitoring of such EMG activities enables neural functional damage to be prevented in, for example, surgeries of thyroid, cervical spine, and craniotomies.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in all its aspects. Although the invention has been described with reference to particular means, materials and embodiments, the invention is not intended to be limited to the particulars disclosed; rather, the invention extends to all functionally equivalent technologies, structures, methods and uses such as are within the scope of the appended claims.

What is claimed is:

1. An endotracheal tube, comprising:
a tubular body having a proximate end portion and a distal end portion, wherein a central passage extends contiguously through the proximate and distal end portions of the tubular body;
an electrode body attached at a plurality of locations thereof to an outside surface of the tubular body, wherein the electrode body extends length-wise from the distal end portion toward the proximate end portion, and
a plurality of electrodes provided on and extending along a length of the electrode body.

2. The endotracheal tube of claim 1 wherein:
the electrode body comprises a sheet of material; and
the sheet of material is wrapped at least partially around the tubular body

3. The endotracheal tube of claim 2 wherein the sheet of material is a flexible polymeric material.

4. The endotracheal tube of claim 1 wherein each of the electrodes:
has an elongated shape; and
extends at least partially along the length of the electrode body.

5. The endotracheal tube of claim 1, further comprising:
a layer of insulating material formed on the electrode body, wherein the layer of insulating material covers a portion of each of the electrodes.

6. The endotracheal tube of claim 5 wherein:
the plurality of electrodes comprises two electrodes extending parallel to the other over at least a portion of lengths thereof;
the layer of insulating material covers the two electrodes; and
a portion of said parallel extending portion of the two electrodes is not covered by the layer of insulating material thereby forming a patient contacting portion of the two electrodes.

7. The endotracheal tube of claim 6 wherein each of the two electrodes:
has an elongated shape; and
extends at least partially along the length of the electrode body.

8. The endotracheal tube of claim 6 each of the two electrodes is formed from a conductive material applied directly onto the electrode body.

9. The endotracheal tube of claim 1 each of the electrodes is formed from a conductive material applied directly onto the electrode body.

10. The endotracheal tube of claim 9 wherein:
the electrode body comprises a sheet of material; and
the sheet of material is wrapped at least partially around the tubular body.

11. An endotracheal tube, comprising:
a tubular body having a proximate end portion and a distal end portion, wherein a central passage extends contiguously through the proximate and distal end portions of the tubular body; and
one or more electrode assemblies each including an elongated electrode body having opposing major surfaces, wherein each of the one or more electrode assemblies has at least one pair of electrodes extending lengthwise on a first one of the major surfaces of the elongated body thereof, wherein the elongated body of each of the one or more electrode assemblies is engaged at plurality of locations along a length of the tubular body, wherein a second one of the major surfaces of the elongated body of each of the one or more electrode assemblies faces an exterior surface of the tubular body, wherein each one of the electrode assemblies includes a layer of insulating material formed on the elongated electrode body thereof, wherein the layer of insulating material covers a portion of each electrode of each one of said at least one pair of electrodes, thereby providing an uncovered portion of the at least one pair of electrodes to serve as a patient contacting thereof.

12. The endotracheal tube of claim 11 wherein:
one electrode assembly is provided;
the elongated electrode body of the one electrode assembly comprises a sheet of material; and
the sheet of material is wrapped at least partially around at least a portion of the tubular body.

13. The endotracheal tube of claim 12 wherein the sheet of material is made from thermoplastic urethane.

14. The endotracheal tube of claim 11 wherein each of the electrodes:
has an elongated shape; and
extends at least partially along the length of the elongated electrode body of a respective one of the one or more electrode assemblies.

15. The endotracheal tube of claim 11 wherein:
one electrode assembly is provided; and
the plurality of electrodes comprises two electrodes extending parallel to the other over at least a portion of lengths thereof.

16. The endotracheal tube of claim 15 wherein each of the two electrodes:
has an elongated shape; and
extends at least partially along the length of the elongated electrode body.

17. The endotracheal tube of claim 16 each of the two electrodes is formed from a conductive material applied directly onto the elongated electrode body.

18. The endotracheal tube of claim 11 each of the electrodes is formed from a conductive material applied directly onto the elongated electrode body.

19. The endotracheal tube of claim 18 wherein:
one electrode assembly is provided;
the elongated electrode body of the one electrode assembly comprises a sheet of material; and
the sheet of material is wrapped at least partially around at least a portion of the tubular body.

20. An endotracheal tube electrode assembly, comprising:
an elongated electrode body having a semi-circular cross-sectional shape defining an endotracheal tube-receiving channel that extends along a length of the elongated electrode body;
a plurality of electrodes provided on a surface of the elongated electrode body outside of the endotracheal tube-receiving channel, wherein the electrodes extend along a length of the elongated electrode body and wherein each of the electrodes is formed from a conductive material applied directly onto the elongated electrode body; and
a layer of insulating material formed on the elongated electrode body covering said electrodes, wherein a portion of each electrode adjacent to each other one of electrodes is not covered by the layer of insulating material thereby forming a patient contacting portion of said electrodes.
